(19) United States
(12) Patent Application Publication
(10) Pub. No.: US 2008/0091119 A1
(43) Pub. Date: Apr. 17, 2008

(54) Assessment and aspiration needle for ovarian follicular fluid

(76) Inventor: Drew V. Moffitt, Tempe, AZ (US)

Correspondence Address: ETHERTON LAW GROUP, LLC, 5555 E. VAN BUREN STREET, SUITE 100, PHOENIX, AZ, 85008, US

(21) Appl. No.: 11/974,241

(22) Filed: Oct. 12, 2007

Related U.S. Application Data
(60) Provisional application No. 60/852,159, filed on Oct. 16, 2006.

(51) Int. Cl. A61B 5/00; A61B 10/00

(52) U.S. Cl. 600/551; 600/576

(57) ABSTRACT

A device and method for assessing characteristics of follicular fluid in vivo comprises an aspiration needle for oocyte retrieval having one or more sensors disposed on its outer cannula wall or embedded in its outer cannula wall for sensing and measuring characteristics of follicular fluid constituents such as pH level and oxygen concentration. The sensors are microsensors that communicate through a physical connection or wirelessly with a meter and display. The measurements are recorded and optionally printed for later review and selection of the most viable oocytes. The method involves inserting into an ovary an aspiration needle with one or more sensors. The needle is guided to a first follicle and inserted in the first follicle. Characteristics of the follicular fluid are sensed and measured and accordingly displayed on the meter and recorded for later reference and selection. The fluid is collected into a first collection container. The sensors can then be reset to a reference baseline and then additional follicles can be aspirated and measured in the same manner. After collection, the measurements of follicular fluid characteristics for each follicle can be assessed and the most viable oocytes can be selected.

### CROSS-REFERENCE TO RELATED APPLICATIONS

This application claims the benefit of co-pending provisional application No. 60/852,159 filed Oct. 16, 2006.

### FIELD OF INVENTION

This invention relates to a device and method for assessing characteristics of follicular fluid in vivo. This invention relates particularly a device and method for assessing such characteristics to enable improved selection of quality oocytes for fertilization during in vitro fertilization and facilitate basic research in ovarian physiology.

### BACKGROUND

In vitro fertilization (“IVF”) involves retrieving eggs from the female's ovary by placing a needle into one or more ovarian follicles and aspirating follicular fluid containing the oocytes. Sperm is added to the oocyte in vitro with the hope that one or more eggs will be fertilized. Once fertilized, one or more fertilized eggs are implanted into the female's uterus. The embryo develops.

A major reason for IVF failure is a high incidence of chromosomal abnormalities in the oocyte. The fertility potential of the oocyte is influenced by the environment that the oocyte is exposed to in the follicle. For example, oocytes from follicles with high perifollicular blood flow have a lower incidence of abnormalities in the chromosomal arrangement within the metaphase II spindle. Sophisticated color and Doppler ultrasound techniques are used to identify follicles with normal and abnormal perifollicular blood flow. Unfortunately, these techniques are not efficient at allowing a definitive identification of the retrieved oocyte as having come from a follicle with good blood flow or low blood flow.

Certain characteristics of the follicular fluid, such as pH, oxygen, and carbon dioxide saturation (partial pressure), have also been shown to affect the oocyte. Another approach has been to evaluate the follicular fluid in the lab once it is aspirated from the female. However, such ex vivo testing introduces variability and uncertainty into the measurements, and again fails to enable a definitive identification of the retrieved oocyte as having come from a follicle with a good environment. It would be advantageous to be able to able to measure certain characteristics of follicular fluid in vivo, such that the environment that each oocyte came from is known.

Therefore, an object of this invention is to provide a device and method for sensing and assessing certain characteristics of follicular fluid in vivo. It is another object to sense and assess pH, oxygen, and carbon dioxide concentrations of a specific follicle. It is yet another object to be able to sense certain characteristics of follicular fluid such that only oocytes from follicles with desired environments are selected for IVF.

### SUMMARY OF THE INVENTION

This invention uses one or more microsensors near the tip of a single lumen or double lumen aspiration needle, or oocyte retrieval needle, to detect and measure desired characteristics of follicular fluid and its constituents in vivo. Preferably pH levels and oxygen concentrations are measured. The sensors are microsensors that are preferably embedded in the outer cannula wall of the aspiration needle or disposed on the outer cannula wall of the needle so that they do not compromise the follicular fluid and oocytes being retrieved. The microsensors communicate through a physical connection or wirelessly with a meter and display. The measurements are recorded and optionally printed for later review and selection of the oocytes. Oocytes from a desirable environment are considered for transfer.

The method involves inserting into an ovary an aspiration needle with one or more sensors. The needle is guided to a first follicle and inserted in the first follicle. Characteristics of the follicular fluid are sensed and measured and accordingly displayed on the meter and recorded for later reference and selection. The measurements can also be printed for review. The fluid is collected into a first collection container. The sensors can then be reset to a reference baseline and then additional follicles can be aspirated and measured in the same manner. After collection, the measurements of follicular fluid characteristics for each follicle can be assessed and the oocytes from the most desirable follicular fluid environment can be selected.

### BRIEF DESCRIPTION OF THE DRAWINGS

FIG. 1 is a schematic illustration of the present invention combined with commercially available equipment used for follicular fluid aspiration.

FIG. 2 is a schematic illustration of the aspirating needle with sensors.

FIG. 3 is an illustration of a commercially-available electrode for sensing pH levels.

### DETAILED DESCRIPTION OF THE INVENTION

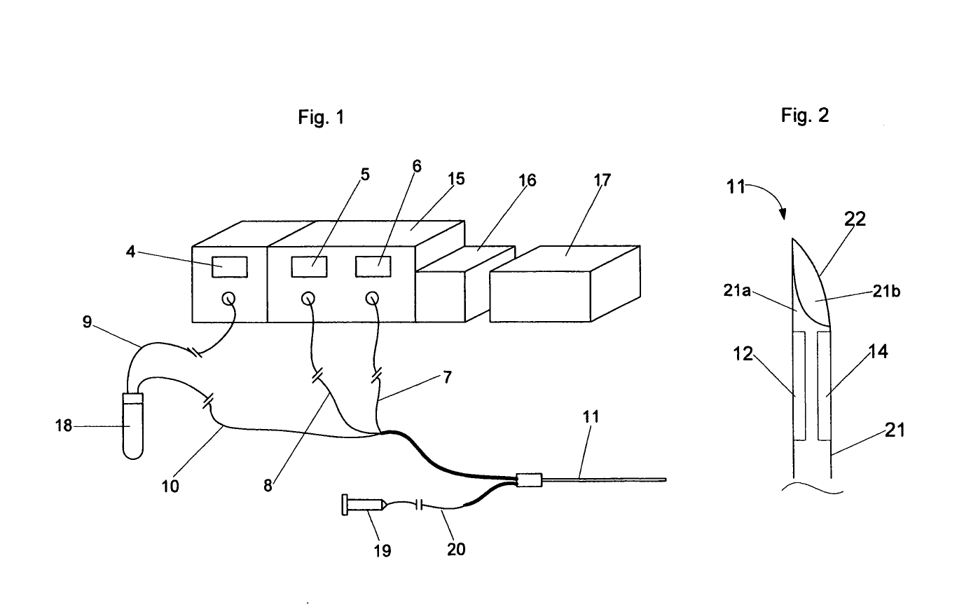

FIG. 1 illustrates the present invention combined with commercially-available equipment used for follicular fluid aspiration. Traditional commercially-available equipment comprises an aspiration needle 11 for oocyte retrieval, either single lumen or double lumen, that is in fluid communication with an aspiration tube or line 10 and optionally a flushing tube or line 20. Aspiration line 10 is in fluid communication at its distal end with aspiration needle 11 and at its proximal end with a collection container 18, such as a test tube. The flushing line 20 is in fluid communication at its distal end with aspiration needle 11 and at its proximal end with a syringe 19 and is used for flushing the follicle during oocyte recovery. Collection container 18 also cooperates with a vacuum tube or line 8 that is controlled by a vacuum regulator such that a vacuum draws follicular fluid and oocytes through aspiration line 10 and into container 18 when aspiration needle is inserted into a follicle and a vacuum is applied. Additional collection containers (not shown) can be substituted for the collection container 18 so that follicular fluids from additional follicles are collected in separate collection containers. For example, follicular fluid from a first follicle is collected in a first collection container, follicular fluid from a second follicle is collected in a second collection container, follicular fluid from a third follicle is collected in a third collection container, and so on.

The present invention adds sensor technology to the traditional aspiration needle and collection system. The aspiration needle 11 of the present invention comprises a cannula having distal and proximal ends, a shaft 21, an outer wall 21a, and an inner wall 21b. The distal end comprises a needle tip 22. The proximal end of the cannula is in fluid communication with the distal end of aspiration line 10 and optionally with the distal end of flushing line 20 as well. Aspiration needles for oocyte retrieval are known in the art and are available commercially. Aspiration needle 11 is a single lumen needle. Alternatively, a double lumen needle can be used. A single lumen needle requires that any irrigation or flushing that is performed be conducted through the same fluid path that is used for aspiration. A double lumen needle has a first fluid path for aspiration and a second fluid path for irrigation or flushing. Preferably 16-20 gauge needles are used, and the tip of each needle preferably has a beveled point and is constructed to reflect ultrasound waves for accurate imaging. For general reference, a 16 gauge needle has a nominal outside diameter of 1.651 mm and a nominal inside diameter of 1.194 mm.

Aspiration needle 11 additionally comprises one or more sensors. FIG. 2 illustrates a first microsensor 12 and a second microsensor 14 disposed on the needle shaft 21. The sensors measure one or more constituents of follicular fluid that may create a desirable oocyte environment and thus play a role in oocyte selection. These constituents include pH levels and oxygen concentrations. Additional follicular fluid constituents that may be measured include carbon dioxide, lactate, urea, total protein, triglycerides, fatty acids, homocysteine, interleukins, Interleukin 6, Reactive Oxygen Species, Lipid Peroxidation, anti-apoptotic molecule, sFas, nitric oxide, TNF-alpha, Hyaluronan, Matrix metalloproteinase-9, cortisol and cortisone, leptin, VEGF, Soluble Fas, thermochemiluminescence assay, lipid peroxidation (LPO), total antioxidant capacity (TAC), homocysteine, antioxidants and reactive oxygen species, fibroblast growth factor (FGF), intracellular adhesion molecule (sICAM-1), redox potential, IGF-I/IGFBP-1, granulocyte colony-stimulating factor, Inhibn A and B, prostaglandin, follistatin, FSH, LH, estradiol, testosterone, androsteindione, DHEAS, 17-OHP, progesterone, insulin, insulin binding growth factor, Neutrophin-Trk, Matrix Metalloproteinases, Phospholipase A2, VEGF, lecithin, cholesterol, betaglycan, Ephrin B1, Prokineticin-1, Interleukin-1, mullerian inhibiting substance, adrenomedullin, ENA-78, leptin Macrophage inflammatory protein 3 alpha, NO, Sodium, potassium, chloride, glucose, beta-hydroxybutyrate, Tumor necrosis Factor—Alpha, insulin-like growth factor-binding proteins, pregnancy associated plasma protein A, betaglycan, Ephrin B1, interleukins, adrenomedullin, reactic oxygen species, PTX3, osmotic potentials, Shingosine 1 phosphate, antiphospholipid antibodies, leptin, soluble leptin receptor, PAI-1 plasminogen actiator inhibitor, T-PA Tissue-type plasminogen activator, carotenoids, retinol, Alpha-tocopherol, transforming growth factor beta 1, resistin, inhibins, antioxidant enzymes, renin, tumour necrosis factor stimulated gene 6 (TSG-6), C-reactive protein, ApoN, Superoxide dismutase, vitamin E, vitamin A, beta-carotene, lycopene, FGF, sICAM-1, SCF, metalloproteinases, Leukemia Inhibitory factor, anti-mullerian hormone, and melatonin.

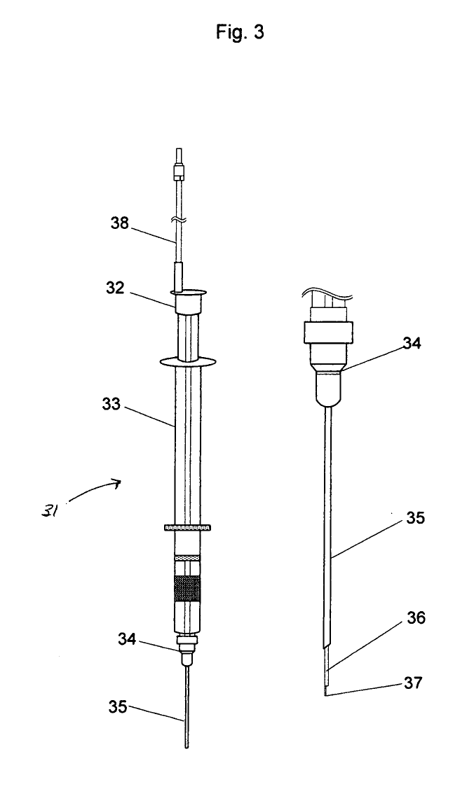

In the preferred embodiment, aspiration needle 11 is equipped with a pH sensor, an oxygen sensor, a carbon dioxide sensor, or any combination thereof. The sensing may be accomplished by measuring chemical, electrical, electrochemical, fluorescent, magnetic, or optical or other EMF reflective wave properties, among others. For example, microsensors that emit light through miniature fiber optics and measure the reflectance are known. See FIG. 3, for example, which illustrates a syringe 31 with a needle 35 and fiber optic sensor 36 for measuring pH. As shown in FIG. 3, syringe 31 includes a plunger 32, housing 33, and needle base 34. The sensor includes a glass fiber 36, sensor tip 37, and fiber cable 38.

To avoid damaging oocytes, preferably sensors 12 and 14, and any additional sensors, are embedded in the cannula 21. Alternatively, the sensors are disposed on the outer surface of the cannula outer wall 21a so that the cannula inner wall 21b and the lumen are not compromised. In another embodiment, the sensors are disposed outside of the cannula and travel substantially in tandem with it, but are not in physical connection to the shaft 21. As used herein, “disposed on” includes sensors embedded, disposed inside, on, outside, or otherwise working in cooperation with the cannula. Preferably passive sensing is used such that the follicular fluid is not affected by the sensing. The sensor may be wireless or physically connected to meter 15 and its displays. A calibration mechanism such as a reference electrode may also be included. Needle microsensors with high spatial resolution and very short response times for these chemicals are known in the art and are available commercially, such as those available from Unisense Medical, Aarhus, Denmark and PreSens—Precision Sensing GmbH, Regensburg, Germany.

The microsensors communicate with meter 15 and displays 5 and 6 to provide and display real-time measurement of characteristics of the follicular fluid. In the preferred embodiment, first sensor 12 senses and measures oxygen concentration and communicates the measurement through a sensor line 7 to meter 15. Also in the preferred embodiment, second sensor 14 senses and measures pH and communicates the measurement through a sensor line 8 to meter 15. The oxygen concentration of the follicular fluid is displayed on display 6 and the pH level of the follicular fluid is displayed on display 5. If additional sensors are included on needle 11, the additional sensors would also communicate with meter 15 and their measurements displayed on additional displays on meter 15. In FIG. 1, the sensors are shown as communicating with meter 15 through physical connections. In an alternative embodiment, meter 15 can include receivers and each sensor can include a transmitter so that the sensors can communicate wirelessly with meter 15.

The microsensors can additionally communicate with a recorder 16 and optionally a printer 17 through a physical connection or wirelessly. The recorder 16 and optional printer 17 record the measurements of the follicular fluid for each follicle for later reference and selection. For example, when retrieving oocytes from a first follicle, the pH level and oxygen concentration of the follicular fluid in the first follicle is recorded and optionally printed on a label, chart, record or some combination thereof. Likewise, when retrieving oocytes from a second follicle, the pH level and oxygen concentration of the follicular fluid in the second follicle is also recorded and optionally printed.

Meter 15 can be combined with the vacuum regulator and include a vacuum display 4. Preferably the overall device is combined with ultrasound imaging to locate each follicle.

The method of assessing and aspirating follicular fluid comprises inserting into an ovary aspiration needle 11 with one or more microsensors 12 and 14 for sensing follicular fluid constituents. In the preferred embodiment, aspiration needle 11 senses pH levels and oxygen concentration in ranges commonly found in follicular fluid, across good and bad environments. The sensors sense at an appropriate threshold, accuracy and precision to accurately evaluate a follicular environment. Using ultrasonic imaging, aspiration needle 11 is guided to and inserted into a first follicle. The pH and oxygen are sensed, measured, displayed, and recorded for each follicle. Preferably the fluid is aspirated into a collection container such as a test tube that is identified or associated with the follicle's characteristics. If desired, the sensors may be reset to a reference baseline before moving to the next follicle.

Once fluid is aspirated, the process may be repeated, guiding aspiration needle 11 to and inserting it into a second follicle. The pH level and oxygen concentration are sensed, measured, displayed, and recorded and the cycle is repeated until all available follicles are aspirated. It is desirable to aspirate all oocytes, regardless of environment, to avoid distressing the patient. The readings are recorded for later analysis and oocyte selection. Preferably the data is recorded in electronic memory as well as printed on paper.

While there has been illustrated and described what is at present considered to be a preferred embodiment of the present invention, it will be understood by those skilled in the art that various changes and modifications may be made, and equivalents may be substituted for elements thereof without departing from the true scope of the invention. Therefore, it is intended that this invention not be limited to the particular embodiment disclosed as the best mode contemplated for carrying out the invention, but that the invention will include all embodiments falling within the scope of the appended claims.

I claim:

1. An assessment and aspiration device comprising:
a. an aspiration needle comprising one or more lumens and an outer cannula wall;
b. a first sensor disposed on the aspiration needle; and
c. a display that communicates with the first sensor.

2. The device of claim 1 further comprising a second sensor disposed on the aspiration needle.

3. The device of claim 1 wherein the first sensor is embedded in the outer cannula wall.

4. The device of claim 1 wherein the first sensor is disposed on the outer cannula wall.

5. The device of claim 1 wherein the first sensor travels substantially in tandem with the aspiration needle.

6. The device of claim 1 wherein the first sensor senses oxygen concentrations in follicular fluid.

7. The device of claim 1 wherein the first sensor senses pH levels of follicular fluid.

8. The device of claim 2 wherein the first sensor senses oxygen concentrations in follicular fluid and wherein the second sensor senses and measures pH levels of follicular fluid.

9. The device of claim 1 wherein the first sensor comprises an electrical microsensor, a chemical microsensor, an electrochemical microsensor, a fluorescent microsensor, an optical microsensor or a microsensor capable of detecting electromagnetic filed reflective wave properties.

10. The device of claim 1 wherein the first sensor comprises a calibration electrode.

11. The device of claim 1 wherein the first sensor communicates with the display through an electrical connection.

12. The device of claim 1 wherein the first sensor comprises a transmitter such that the first sensor communicates with the display wirelessly.

13. A method of assessing and aspirating follicular fluid comprising
a. inserting into an ovary an aspiration needle comprising one or more sensors;
b. guiding the aspiration needle to a first follicle and inserting it into the first follicle;
c. sensing, measuring, and recording one or more follicular fluid constituents of the first follicular fluid of the first follicle; and
d. aspirating the first follicular fluid into a first container.

14. The method of claim 13 further comprising resetting the sensors to a reference baseline after aspirating the first follicular fluid.

15. The method of claim 14 further comprising:
a. guiding the aspiration needle to a second follicle;
b. inserting the aspiration needle into the second follicle;
c. sensing, measuring, and recording one or more follicular fluid constituents of the second follicular fluid of the second follicle; and
d. aspirating the second follicular fluid into a second container.

16. The method of claim 13 wherein the aspiration needle is guided with ultrasound technology.

17. The method of claim 13 wherein the aspiration needle comprises a first sensor that senses and measures oxygen concentrations and a second sensor that senses and measures pH levels and wherein the method further comprises sensing, measuring and recording the oxygen concentration and pH level of the first follicular fluid of the first follicle.

18. The method of claim 15 wherein the measurements of follicular fluid constituents are recorded in electronic memory.

19. The method of claim 15 wherein the measurements of follicular fluid constituents are recorded in printed form.

20. The method of claim 15 further comprising selecting an oocyte for fertilization from the first follicular fluid and second follicular fluid.
